(19) United States
(12) Patent Application Publication
(10) Pub. No.: US 2017/0252210 A1
(43) Pub. Date: Sep. 7, 2017

(54) THERAPEUTIC EYE MASK SYSTEM

(71) Applicant: BRUDER HEALTHCARE COMPANY, LLC, Alpharetta, GA (US)

(72) Inventor: Mark H. BRUDER, Alpharetta, GA (US)

(73) Assignee: BRUDER HEALTHCARE COMPANY, LLC, Alpharetta, GA (US)

(21) Appl. No.: 15/443,199

(22) Filed: Feb. 27, 2017

Related U.S. Application Data
(60) Provisional application No. 62/301,999, filed on Mar. 1, 2016.
(60) Provisional application No. 62/430,430, filed on Dec. 6, 2016.

(51) Int. Cl. A61F 9/00; A61F 7/02; A61F 9/04

(52) U.S. Cl. CPC A61F 9/0026; A61F 9/045; A61F 7/02; A61F 2007/0004

(57) ABSTRACT

A therapeutic mask for treatment of the eyes, including an eye coverage portion for application to the eye, a receiver in the eye coverage portion positioned to align with the eye, and at least one pod for detachably securing within the receiver. The pod includes material for delivering thermal, moisture and/or medication therapy and treatment to the eye.

### CROSS-REFERENCE TO RELATED APPLICATIONS

This application claims priority to U.S. Provisional Patent Application Ser. No. 62/301,999 filed Mar. 1, 2016 and U.S. Provisional Patent Application Ser. No. 62/430,430 filed Dec. 6, 2016, both of which are hereby incorporated herein by reference in its entirety.

### TECHNICAL FIELD

The present invention relates generally to the field of ophthalmic medical therapy or palliative care of the eye. More particularly, the present invention relates to a mask or compress for thermal treatment and/or delivery of medication to the eyes, sinus and facial areas of a subject or patient.

### BACKGROUND

Various conditions of the eye may require medical or palliative care. For example, blepharitis is a common and ongoing condition where the eyelids become inflamed (swollen), with oily particles and bacteria coating the eyelid margin near the base of the eyelashes. This condition causes irritation, itchiness, redness, dry eye and stinging or burning of the eyes. While the underlying causes of blepharitis are not completely understood, it can be associated with a bacterial eye infection, symptoms of dry eyes or certain types of skin conditions such as acne rosacea. Anterior blepharitis affects the outside of the eyelid where the eyelashes are attached. This can be caused by bacterial (or sometimes viral) infection. If left untreated, anterior blepharitis can lead to thickened and inward-turned or outward-turned eyelids and even vision problems from in-turned eyelashes damaging the cornea. Posterior blepharitis is a condition that results from a dysfunction of the eye's tiny oil glands (meibomian glands) in the eyelids at the base of the eyelashes. When meibomian glands become clogged from posterior blepharitis, it can cause a stye or chalazion to form. Posterior blepharitis can also lead to thickened eyelid margins and crusty eyelids.

An estimated 40.9 million people in the United States aged 18 or older wear contact lenses. The International Workshop on Contact Lens Discomfort, published in 2013, put forth dryness of the eyes as a primary reason for contact lens intolerance. When a contact lens is place on the eye, the tear film structure becomes altered resulting in a pre-lens thinned lipid layer and a post-lens thinned aqueous layer. As a result of this disruption from the contact lens, the tear film tends to have an increased rate of evaporation leading to poor wetting on the surface of the contact lens and inadequate lubrication on the surface of the eye. This is further exacerbated if the patient has an already unstable lipid layer due to the presence of meibomian gland dysfunction (MGD). MGD is considered by many to be the leading cause of dry eye disease throughout the world, and is a chronic and progressive condition that can contribute to a poor quality lipid layer and lead to contact lens discomfort. Contact lens wearers often report dry eye symptoms and show signs of MGD including gland atrophy, thinned lipid layer, and increased tear film instability. It has been shown that in many patients with intolerance to contact lenses, MGD has been observed. Therefore, treatment of MGD may support functioning of the meibomian glands and lead to improvement in patient contact lens comfort.

Hygienic home treatment of such ocular disorders can be a two-step process. First, the patient softens the debris and scurf that accumulates around the eye. The debris can be softened by applying a warm compress, diluted baby shampoo, or a specialized liquid solution to the eyelid margin. This first step is intended to prepare the debris for removal while preventing further irritation to the eye. Second, the patient can attempt to remove the debris by physically scrubbing the eyelid margin, the base of the eyelashes, and the pores of the meibomian glands. This scrubbing is routinely attempted with either a generic cotton swab, a fingertip, or a scrub pad placed over the fingertip and applied against the eye. By cleaning debris and scurf free from the base of the eyelashes and unclogging the pores of the meibomian glands, the patient may improve the overall health of the eyelid margin; thereby reducing irritation, burning, and other symptoms related to the disorder.

Thermal therapy can also be used for medical or palliative care of a human or animal subject or patient, for example by delivering moist heat or cold to the eye region. In example applications, thermal therapy can be used to unblock glands in the eye to help treat dry eye. Moist heat may also be used to help reduce elevated intraocular pressure to either treat or help prevent open-angle glaucoma. Delivery of medications to the eyes, such as for treatment of blepharitis may be enhanced by application of thermal therapy in combination with the medication. Applying heat to the inner eyelid may also help safely remove gland obstructions and stagnant gland content.

Many currently known eye treatment masks are not designed to securely fit the eye, causing issues in some forms of therapeutic treatment. For example, when a patient uses a continuous positive airway pressure (CPAP) machine for treatment of sleep apnea, air can sometimes blow back from the mask of the CPAP machine into the user's eyes, causing dryness of the eyes. Known eye masks and eye compresses may not fit securely to the eye and have not been found entirely successful in protecting the eyes from this blow-back dryness.

Needs exist for improvements to ophthalmic medical therapy or palliative care of the eye. It is to the provision improved therapeutic eye mask system and treatment methods meeting these and other needs that the present invention is primarily directed.

### SUMMARY

In example embodiments, the present invention provides an eye mask or therapeutic eye compress device for delivering thermal therapy such as moist heat or cold to at least one eye or eye region of a human or animal patient or subject. The therapeutic device includes at least one eye coverage portion for application over and to the eye and/or eye region. The mask optionally includes at least one receiver positioned to align with the at least one eye and fit securely to the patient's eye region. The mask optionally also includes at least one pod detachably secured within the at least one receiver, incorporating material for delivering moist heat therapy or cold treatment to the eye, and optionally to deliver topical medication to the eye region. The therapeutic device optionally includes antimicrobial treatment to combat bacterial and fungal growth.

In example embodiments of the invention, the therapeutic device includes pods having a granular fill material such as a hydrophilic zeolite or molecular sieve material, optionally loaded with at least one metallic or other antimicrobial agent, such as for example a silver, copper, copper oxide, gold, magnesium oxide, aluminum oxide, titanium dioxide, zinc oxide, cobalt, nickel, zirconium, molybdenum, tin, lead and/or other metals, metal oxides, metal ions, metal particles or nanoparticles, and alloys, mixtures or combinations thereof. In example embodiments, the antimicrobial agent is retained in the therapeutic device over multiple uses.

In another aspect, the invention relates to an eye mask article or compress system comprising at least one eye coverage portion for application to at least one eye. The article includes a mask with at least one eye coverage portion for application to the at least one eye. The mask includes at least one indented pocket positioned to align with the at least one eye. The mask also includes at least one thermal treatment delivery pod detachably secured within the at least one indented pocket. The at least one pod includes material for delivering moist heat therapy and treatment to the at least one eye. The mask optionally further comprises medicament dispensed toward the at least one eye by the at least one pod.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description are exemplary and explanatory of preferred embodiments of the invention, and are not restrictive of the invention, as claimed.

### BRIEF DESCRIPTION OF THE DRAWINGS

FIG. 1 is a perspective view of a therapeutic eye mask system according to an example embodiment of the invention.

FIG. 2 shows a back view of the therapeutic eye mask system depicted in FIG. 1.

FIG. 3 shows the therapeutic eye mask system depicted in FIG. 2 including detachable pods.

FIGS. 4A-C show a front, back and side view of the therapeutic eye mask system of FIG. 1.

FIG. 5 is a perspective view of a therapeutic eye mask system according to an example embodiment of the invention.

FIG. 6 shows a back view of the therapeutic eye mask system of FIG. 5.

FIGS. 7A-C show a front, back and side view of an eye mask portion for use in a therapeutic eye mask system according to an example embodiment of the invention.

FIGS. 8A-C show a front, top and side views of a therapeutic eye mask system with the eye mask portion of FIGS. 7A-C.

FIG. 9 shows the therapeutic eye mask of FIG. 1 worn by a human patient.

FIG. 10 shows the therapeutic eye mask of FIGS. 8A-C worn by a human patient.

FIGS. 11A-B show the therapeutic eye mask of FIGS. 8A-C worn by a human patient.

FIG. 12 shows a therapeutic eye mask system according to another example embodiment of the invention.

FIG. 13 is an exploded view of a therapeutic eye mask system according to another example embodiment of the invention, having interchangeable pods.

FIG. 14 shows a detailed exploded view of the therapeutic eye mask system of FIG. 13.

FIG. 15 shows a detachable pod for use in a therapeutic eye mask according to an example embodiment of the invention.

FIG. 16 shows a detachable pod according to another example embodiment of the present invention.

FIG. 17 shows detachable pods of varying shapes according to further example embodiments of the invention.

FIG. 18 shows detachable pods of varying sizes according to example embodiments of the invention.

FIG. 19 shows a therapeutic eye mask system according to an example embodiment of the invention.

FIG. 20 shows a therapeutic eye mask system according to an example embodiment of the invention.

FIG. 21 is a side view of a therapeutic eye mask system according to an example embodiment of the invention.

FIG. 22 shows a front view of the therapeutic eye mask system of FIG. 21.

FIG. 23 shows an alternative configuration of the therapeutic eye mask system of FIG. 22.

### DETAILED DESCRIPTION OF EXAMPLE EMBODIMENTS

The present invention may be understood more readily by reference to the following detailed description taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

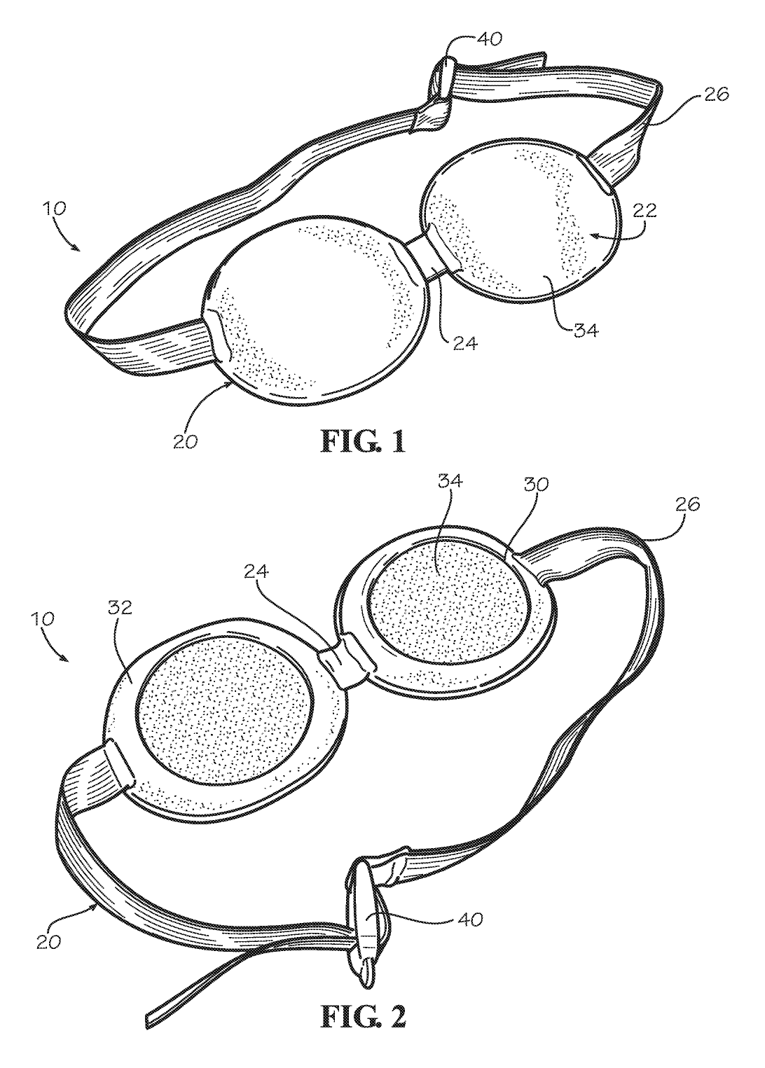

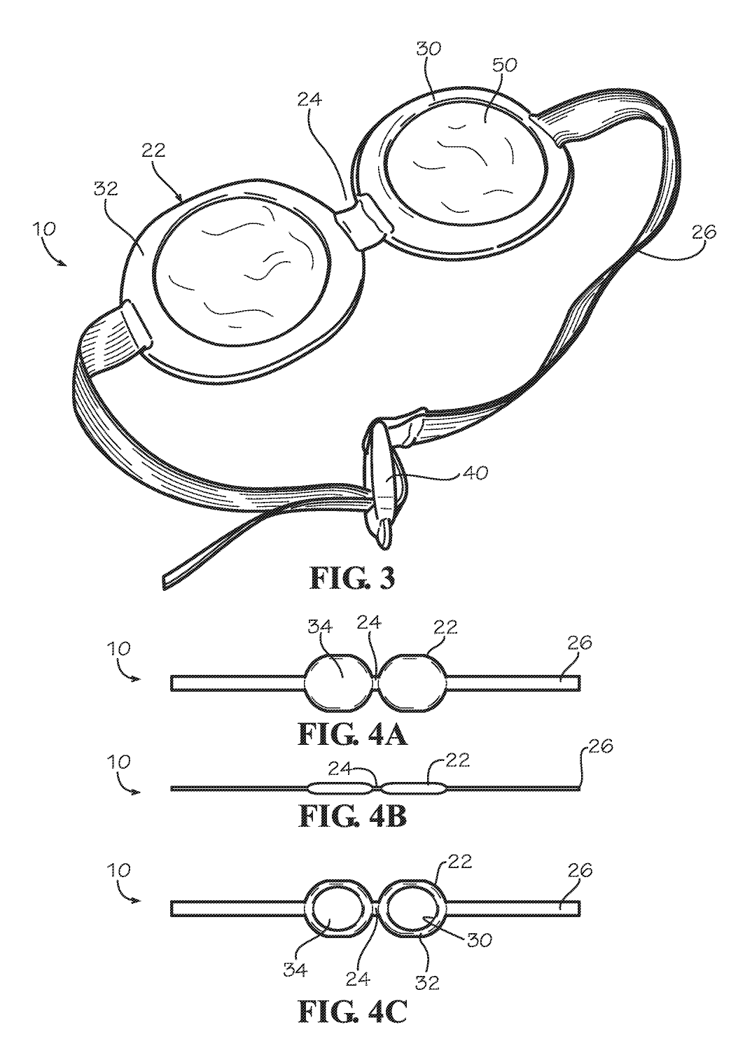

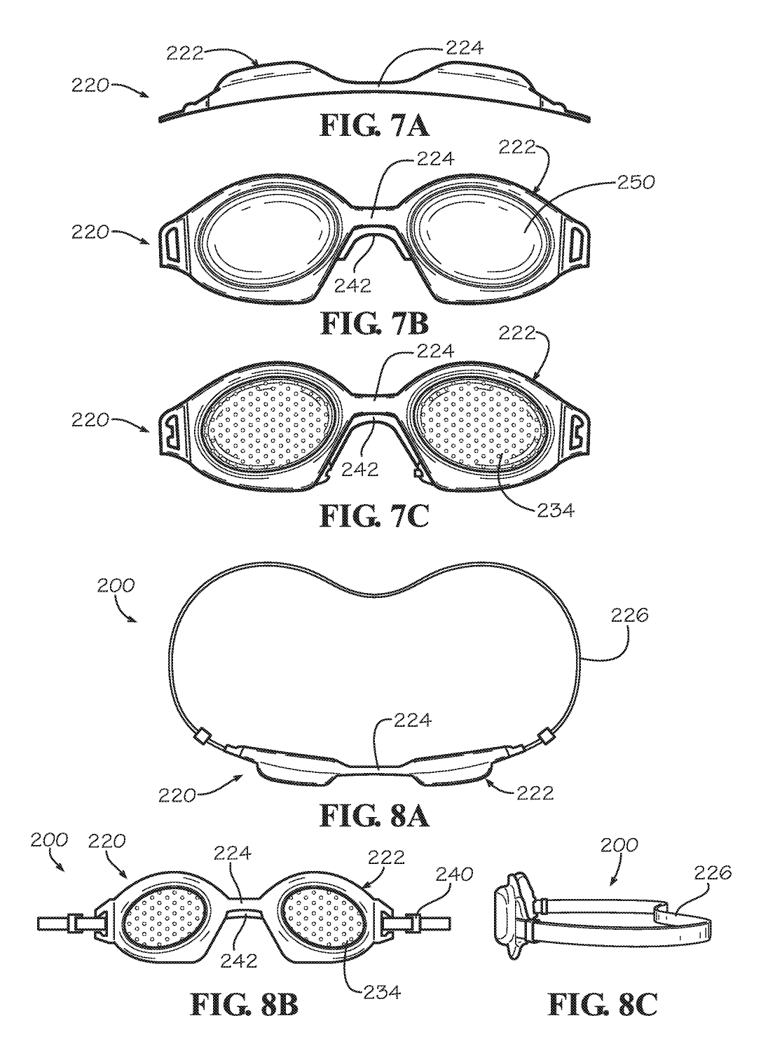

With reference now to the drawing figures, wherein like reference numbers represent corresponding parts throughout the several views, FIGS. 1-23 show a therapeutic eye mask system according to example embodiments of the present invention. The therapeutic eye mask system 10, shown in FIGS. 1-4, generally includes an eye mask 20 and at least one detachable therapy pod 50. The eye mask 20 can include two separate eye coverage portions 22 connected by a nose bridge 24 and a securing strap 26. The mask 20 is configured to be worn on the head of a human patient with one of the eye coverage portions 22 positioned over each eye of the patient. In example embodiments, the securing strap 26 is positioned around the user's head to hold the eye coverage portions 20 against the eyes of the user.

In example embodiments, the mask 20 can have a monolithic uniform construction, or may be separate pieces fastened together, for example by stitching, adhesive, fasteners or other attachment means. In example embodiments, the mask 20 is constructed of a lightweight and durable material. The mask 20 can be made of a soft and flexible material, for example foam or polyester. It can be constructed of perforated thermoformed foam to improve breathability. The mask 20 can be constructed from laminated foam or soft-flexible, open-cell foam with polyester fabric. In particular examples, the mask 20 is constructed from a 2 lb./ft³, ⅜″ thick polyether polyurethane foam, flame laminated to matte black polyester fiber interlock fabric on both sides. Alternatively, the mask 20 is constructed from polyester, rayon, spandex, silk or other natural and/or synthetic fabrics or materials. The mask material can optionally be selected to have insulative or heat-transmissive properties to affect the temperature transferred from the mask to the patient's eyes, ensuring safety. The material may optionally be washable for reuse, or alternatively can be a single-use disposable product. In example embodiments, the mask 20 may be constructed of a material containing nanobeads comprising an antimicrobial metal.

The mask 20 depicted in FIGS. 1-4 includes two eye coverage portions 22. The eye coverage portions 22 are designed to be independent structures such that each can independently conform to the respective eye region of the patient. The eye coverage portions 22 can each include a receiver 30 for holding a detachable therapeutic pod 50. The receivers 30 are configured to hold position the pods 50 over the patient's eyes when the mask 20 is worn. In example embodiments, the receivers 30 are permanently-formed indents or pockets in the eye coverage portion 22. For example, the indents or pockets can be positioned on the side of the mask that directly faces the eyes of the patient when worn. In use, two removable pods 50 are received in the mask 20, one within each of the receivers 30. Each eye coverage portion 22 can optionally include an eye cushion 32 and an eye cover 34. The eye cushion 32 is configured to surround the receiver 30 and provide padding on a portion of the mask 20 that abuts the users face when worn. The eye cover 34 is configured to cover the outside of the eye coverage portion 22. In alternative embodiments, the mask 20 does not include an eye cover 34 and the eye coverage portion 22 is formed from a circular frame that is open when a detachable pod 50 is not in the receiver 30. The eye coverage portions 22 can be a substantially round shape as depicted in FIGS. 1-4, or can be an oval, elliptical, polygonal, angled or other non-round shape.

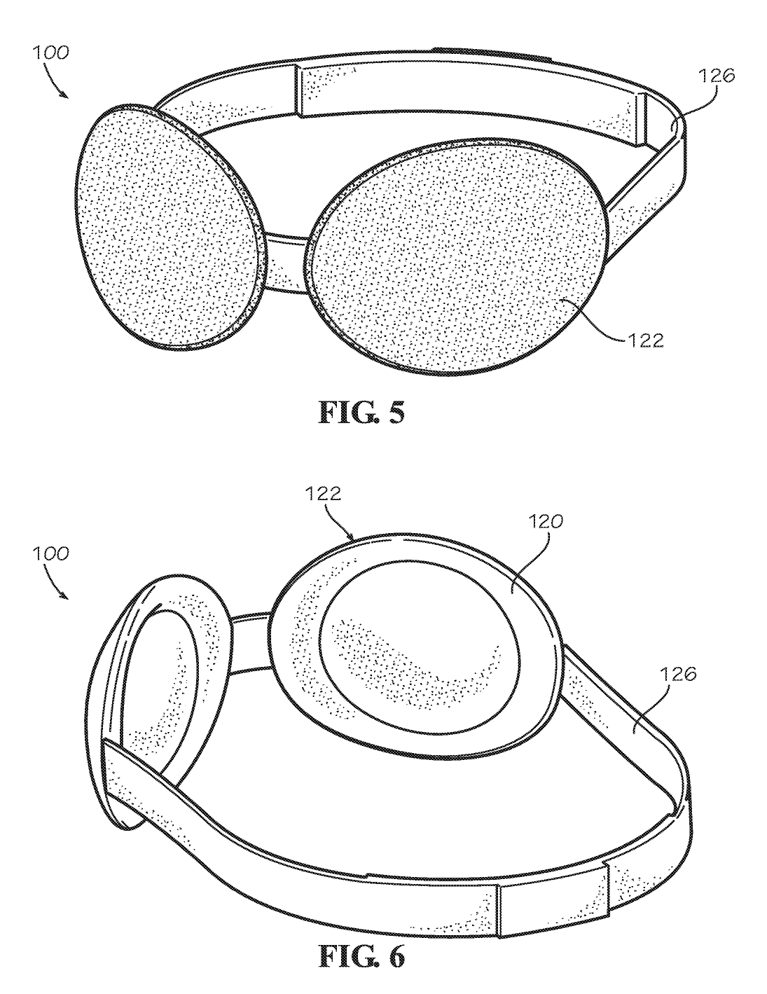

The eye coverage portions 22 can be connected by a nose bridge 24 and a securing strap 26. The nose bridge 24 can be formed from a flexible and/or elastic material that allows the nose bridge to fit the face shape of a variety of users. In alternate embodiments, the nose bridge 24 is adjustable. In the depicted embodiments, the securing strap 26 is formed from a strap extending between the eye coverage portions 22. The securing strap 26 can include an adjustment mechanism 40 that allows the user to adjust the length of the strap. In alternate embodiments of the eye mask 100, depicted in FIGS. 5 and 6, the securing strap 126 is formed from two straps, each coupled at a first end to an eye coverage portion 122. The second end of each securing strap 126 is configured to be removably coupled to the other securing strap. The attachment mechanism can include snaps, ties, hook-and-loop fasteners or other releasable attachment mechanisms. The nose bridge 24 and securing strap 26 are configured so that the eye mask 20 can be one size to fit all or most users. Alternatively, the eye mask 10 can be produced in different sizes. The securing strap can also include a comfort wrap (not pictured). In alternate embodiments, the mask 20 can include dual supports to fit over the ears in place of the securing strap 26.

In other embodiments, the mask portion 220 of the therapeutic eye mask system 200 is shaped similar to swimming goggles. The eye coverage portions 222 have a tear drop shape with the top and bottom having a curved shape and the side adjacent the nose bridge being straight so as to follow the angle of the nose. The detachable pods 250 have a generally oval shape and are angled relative to the nose bridge 224. The nose bridge 224 can include a padded portion 242 to provide comfort when worn. The eye covers 234 can be perforated for breathability. The securing strap 226 of the depicted embodiment includes two adjustment mechanisms 240, one adjacent to each of the eye coverage portions 222.

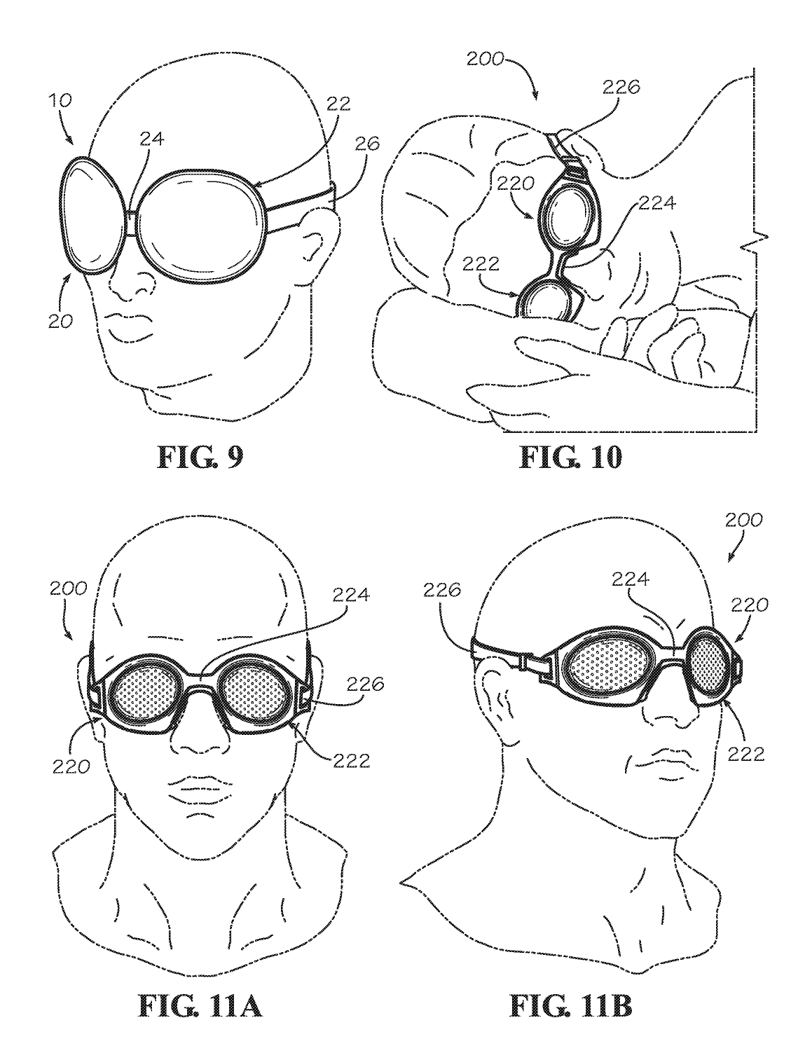

The mask 20, 220 portion of the therapeutic eye mask system 10, 200 is designed to fit securely to the patient's eye region with an eye coverage portion 22, 222 over each eye, the nose bridge 24, 224 positioned across the patient's nose and the securing strap 26, 226 extending around the back of the patient's head, as shown in FIGS. 9-11. The eye mask 20, 220 is designed such that it can be safe and comfortable to wear while sleeping, as shown in FIG. 10. The secure fit can also prevent air from being blown into the eye while the mask is being worn, for example when worn in combination with a CPAP machine. In example embodiments, the dual eye coverage portion design allows full conformance within the eye socket. The design also optionally allows heat and moist therapy to reach the sinuses to help relieve sinus pressure.

As discussed above, the eye coverage portions 22 of the mask 20 each include receivers configured to releasably secure a therapy pod 50. In example embodiments, the pods 50 have a soft outer shell construction that is able to contain a fill material capable of delivering therapy treatment, for example moist heat and cold treatments. The outer shell of the pods 50 can be formed of fabric, non-wovens or other natural or synthetic materials, and is preferably thermally and moisture transmissive, to allow heat and/or moisture to pass through the shell to and from the fill material. Example fill materials include hydrophilic zeolite granules or particles, and optionally silver or other antimicrobial treatments, and/or other materials. The fill material can be loosely contained and held within the pod 50 such that each pod will conform to the eye area of the patient when worn. Alternatively a liquid or gel fill material can be used. The pods 50 can be designed for single use or can be washable and re-usable.

In example embodiments, the fill material contained within the pods comprises a synthetic porous crystalline granular aluminosilicate zeolite, for example a hydrophilic natural or synthetic zeolite, also referred to as a molecular sieve material, or other substances with similar properties. The fill material may further comprise other inert additives and physical matrices without affecting the antimicrobial and hydrous efficacies of the fill. The hydrophilic zeolite granules or beads are configured to repeatedly absorb and release moisture without substantially changing shape or form. Optionally, the pods comprise a granular fill material such as activated alumina, silica gel, bentonite or hydrophilic zeolite or molecular sieve material. In alternate embodiments, the pods comprise capsules or packets of non-granular material (e.g., gel, liquid), powder, or other materials. The pods or granules contained in the pods optionally also contain a metallic or other antimicrobial agent, such as for example a silver, copper, copper oxide, gold, magnesium oxide, aluminum oxide, titanium dioxide, zinc oxide, cobalt, nickel, zirconium, molybdenum, tin, lead and/or other metals, metal oxides, metal ions, metal particles or nanoparticles, and alloys, mixtures or combinations thereof deposited therein. For example, silver or other metal loading of the fill may be attained by the process of ion-exchange. In this process, a solution containing atomic silver or a composition of silver bathes or is passed through a bed of the fill granules. An ion-exchange column method may be performed in which an aqueous solution containing atomic silver or a composition of silver may be passed through a column bed of the fill granules, and the eluted solution may again be passed through the bed or may receive additional silver and then be again passed through the bed.

Various ion-exchange schedules as known in the art may be applied to produce retention of the silver or other metals in the fill material of the pods. For example, the final content by weight of an atomic silver or silver composition, or other metals or antimicrobial agents, may be as high as twenty percent of the final loaded fill granules. In example embodiments, the loaded fill granules produced by ion-exchange will exhibit high retention of the silver or other metals even under subsequent exposure to fluids and microwave irradiation. The fill granules may comprise a blend of both metal loaded and unloaded (i.e., not containing metal) zeolite or other substance(s) retaining silver or other metals. The presence of the atomic silver or other metals preferably will not interfere with the useful properties of the fill granules such as the moisture desorption and adsorption properties which may be desirable in the use of the eye mask or compress system. The inherent hydrophilic nature of example forms of zeolite fill materials provides that substantial water content is available therein by absorption from the atmosphere. The water so absorbed may be sufficient for moist heat delivery applications, or may be supplemented by manually added water, for providing a microwave responsive water content of the eye mask or compress system. The compositions of silver or other metals used may include but are not limited to, metal compounds, and metal salts such as silver chloride and silver nitrate.

The presence of the silver or other metals within the fill granules optionally contained in the pods of the invention provides anti-microbial properties to the therapeutic eye mask system. The ion-exchange loaded fill granules will preferably retain the silver or other metals despite microwave heating as may be required in the use of the eye mask or compress system. Further, the retention of the silver or other metals within the fill granules provides assured antimicrobial performance in a reusable and potentially washable, if so desired, moist heat therapy compress. In other embodiments, the silver or other metals are incorporated into the cover material of the pods, the eye coverage portions, and/or other portions of the eye mask system, in addition to or instead of the fill granules. Alternatively, one or more non-metal antimicrobial materials and/or medications may optionally be incorporated into the fill material, the pods, the eye coverage portions, and/or other portions of the eye mask system.

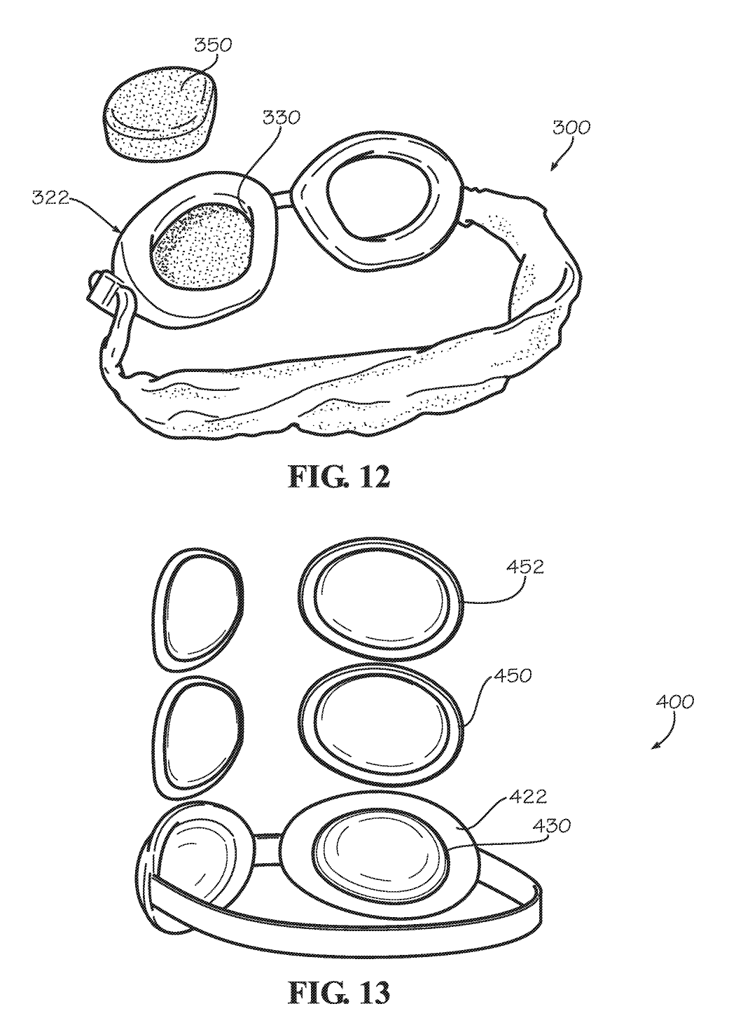

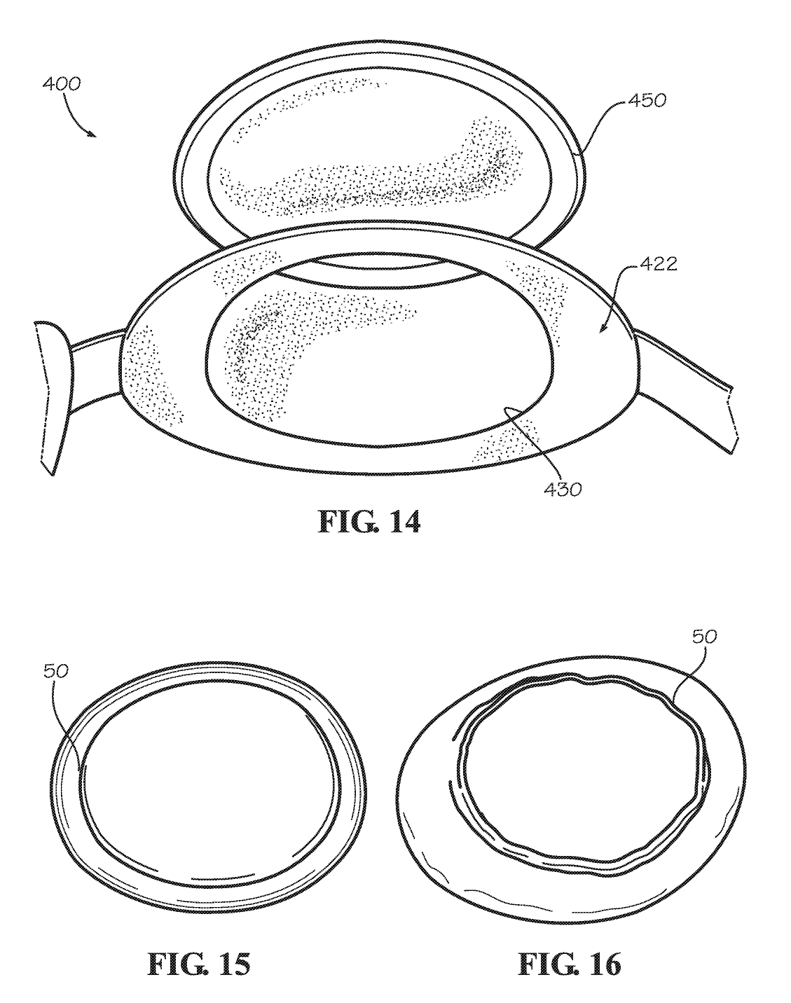

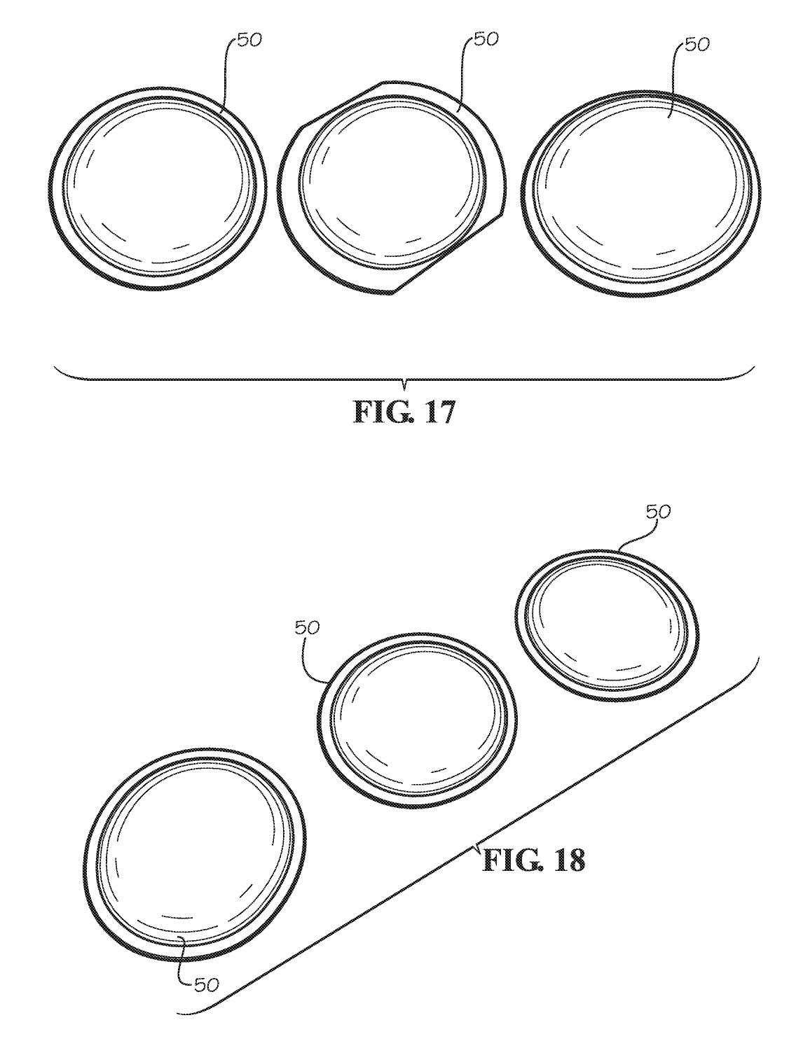

In example embodiments of the therapeutic eye mask system 300, the pods 350 are push fitted into the receivers 330 in each eye coverage portion 322, as show in FIG. 12. The pods 350 can be detachably secured within the receivers 330 through the use of an attachment mechanism, for example a hook-and-loop fastener material secured to both the pods and the receivers, by friction fit, or otherwise. Alternatively, the pods 350 are secured with a positive lock, clip, or a rigid snap. In other embodiments of the therapeutic eye mask system 400, the pods 450 can be slid into the receivers 430 through an opening in the top of the eye coverage portion 422, as shown in FIG. 14. The pods 50 may be round so that they are self-orienting when attached or inserted into the mask, or can be oval, elliptical, polygonal, angled or other non-round shape, as shown in FIG. 17. The pods can be provided in one or more different colors, for example color coded based on their function. For example, a pod for moist heat treatment may be red and a pod for cool treatment may be blue. The pods 50 can also be provided in different sizes, as shown in FIG. 18. In example embodiments, the material and configuration of the mask forms a seal against the wearer's face surrounding the eye area to prevent external airflow from drying the wearer's eyes or drying the pods or medication components positioned within the mask during use.

In use, the entire mask with the pods attached, can be heated or cooled prior to use to provide therapy. The mask can be placed in the microwave to be heated or in the freezer to be cooled. Alternatively, the pods alone can be heated or cooled detached from the mask, then placed in the mask when they reach the desired temperature for treatment.

Additionally, various medicaments can be applied and used with respect to the pods. For example, one or more ophthalmic medications can be infused or injected into the formulation in the pods. Examples of medicants or therapeutic materials capable of delivery using the therapeutic device according to example forms of the invention include a jojoba formulation for treatment of the symptoms of dry eye, steroids such as clobetasol propionate, betamethasone dipropionate, amcinonide or loteprednol etabonate for treatments of diseases of the eyelid, such as chatazion, blepharitis or meibomian gland dysfunction. Medicants may also comprise a dietary or nutritional supplement composition comprising an effective amount of omega-3 fatty acids for treatment of dry eye or meibomianitis. Medicants may also comprise tetracycline, corticosteroids, androgens or androgen analogues. The medicant can also comprise a topical treatment to elevate the side effects of chemotherapy, including eyelash loss.

In alternative embodiments, the therapeutic eye mask system 400 may be combined with a heat transmissive pad or lid scrub or disc 452 that is applied to the skin surface on the eyelid and around the eye, as shown in FIG. 13. In example embodiments, the pad or disc is constructed of non-woven material and optionally a material that can be RF or thermally sealed to hold an antibacterial or other medication. The pad or disc 452 can be impregnated with medication and can be effective in either a moist or dry condition. The antibacterial medication can include, for example, liposomes and/or microspheres. The pad or disc 452 can be removably secured to the compress for single use or multiple uses. The pad can also be impregnated with materials to improve the aesthetics of the eye, like Vitamin E.

In example embodiments, the medication pad or disc 453 is moist-heat transmissive, and application of moist-heat activates the release of the impregnated medication from the pad 452 onto the skin surface of the eyelid or other tissue in or around the user's eyes. In example forms, the pad or disc is constructed to prevent the impregnated medication from passing back into the compress during application. For example, a one-way sheet barrier material can be placed between the pad or disc and the compress to prevent any antibacterial medication from the disc from entering the compress.

In example embodiments, the medication pad or disc 452 can have a round or oval disc shape with appropriate size and shape to be placed over a single eyelid or attached to a single eye coverage portion 422. In use, two pads or discs can be used, one attached to each eye coverage portion 422. A plurality of discs can be stored within a container containing antibacterial medication fluid, so as to pre-moisten the discs during storage. In an example manner of use, two discs are removed from the container and one is placed over each eye of a patient while the patient is lying down and the mask is placed over the discs so that the discs are held in between the eye and the eye coverage portion. Alternatively, the disc 452 may be attached to the detachable pod 450, such that when the eye mask or compress is worn, the disc rests in between the eyelid and the detachable pod. In other embodiments, the disc 452 is placed in the receiver 430 with the detachable pod 450.

In other example embodiments, the medicated disc is used as an eye lid cleaning or treatment wipe. The medicated disc is impregnated with dry specialized formula that is activated by the heat and moisture from the mask. The medication assists in preparing debris in the eye for removal. The disc can then be removed from the mask and used to scrub or wipe the eye area, removing the debris. The disc can be formed of a scrim or non-woven material that accepts dry impregnation of specialized formulas or medication. Ideally, the medicated disc allows the passage of moisture and heat. The medicated disc can remove oil debris and pollen from the eye lids and enhance the moisture of the skin around the eye. The disc can also protect the mask itself from make-up or other contaminates.

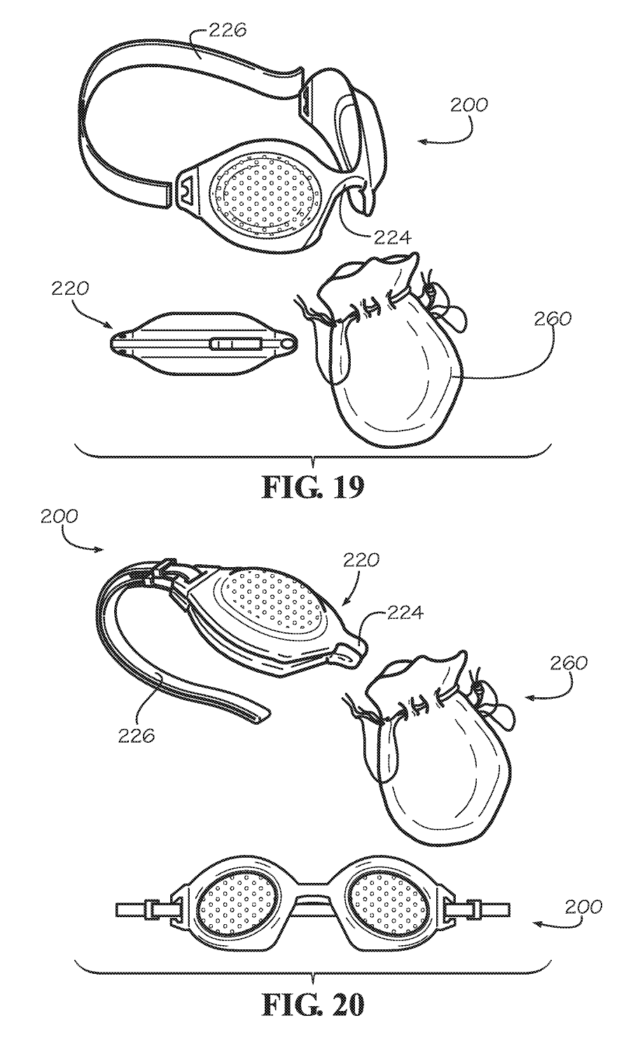

The therapeutic eye mask system 200 can also include a storage bag 260, as shown in FIGS. 19 and 20. In the depicted embodiment the mask portion 220 is configured to fold at the flexible nose bridge 224 to fit within the storage bag 260. The securing strap 226 can be detached from the mask 220 for storage, as shown in FIG. 19, or the mask 220 can be stored with the securing strap 226 attached, as shown in FIG. 20.

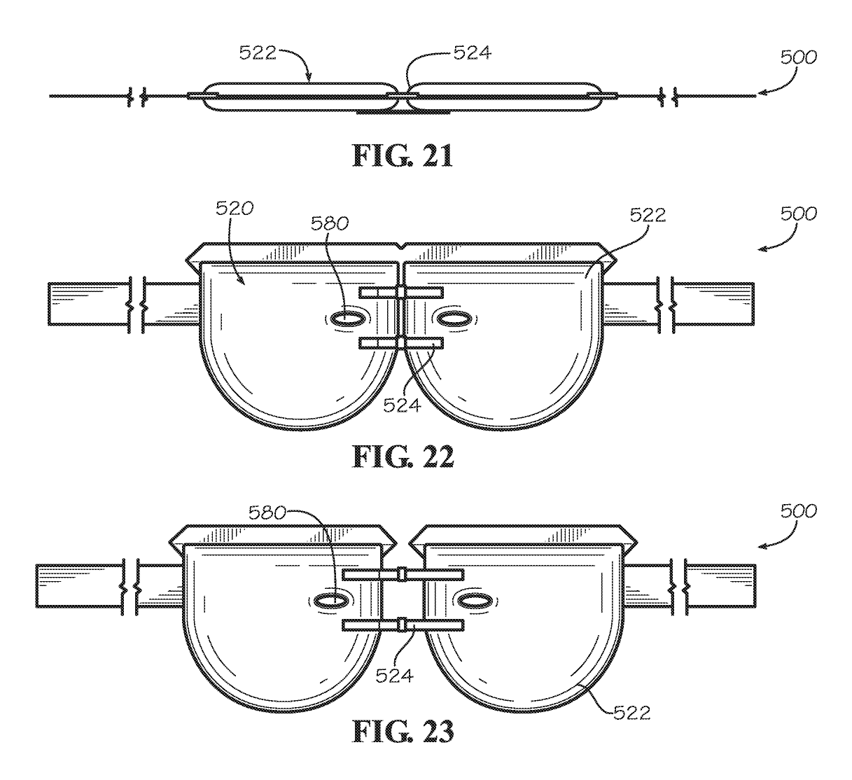

FIGS. 21-23 show a mask 500 according to another example embodiment of the present invention. In the depicted embodiment, each eye coverage portion 522 includes an opening, recess, depression, slit or other void 580 configured to be positioned over the wearer's corneas when the mask is worn. Temperatures over 39.5° Celsius may be unsafe for the cornea. The openings or voids 580 positioned over and around the cornea can help prevent application of excess heat on and around the cornea, preventing conditions such as corneal warping, while allowing moist heat therapy to reach the eyelid and surrounding eye area. In example embodiments, the cornea voids 580 are around 15 mm wide. In example embodiments, the voids 580 can be recesses in the eye coverage portion 522 or detachable pods or alternatively can be slits or openings extending through the entire thickness of the eye coverage portion and detachable pods whereby the user can see through the openings when worn. In alternate embodiments, the eye coverage portions or detachable pods can include a heat insulating material applied over one or more portions of the eye covers configured to be positioned over the corneas when in use. For example, an insulating shield can be attached to the central regions of each eye cover 522 or detachable pod. The insulating shield is configured to vault the cornea and prevent application of excess heat on or around the corneas. This embodiment can further include an adjustable, flexible nose bridge 524. The adjustable nose bridge 524 allows the user to adjust the spacing distance between eye coverage portions 522 and/or between the voids in each eye cover to accommodate varying distances between human eyes from one individual to the next. The average pupillary distance for a human is around 57 mm to 65 mm. In example embodiments the adjustable nose bridge 524 can be changed to accommodate a distance between cornea areas ranging from about 45 millimeters to about 74 millimeters. This range is sufficient to accommodate the eye placement of the majority of adult and/or child male and female humans. In example embodiments, one or more adjustable or expandable straps or bands are provided between the eye covers to allow adjustment of the relative positions or spacing of the eye covers. Alternatively, the eye covers can be detachably mounted to the retention strap, for example with hook-and-loop fastener material, to allow for reposition of the eye covers on the strap to vary the spacing and position of the eye covers.

While the invention has been described with reference to preferred and example embodiments, it will be understood by those skilled in the art that a variety of modifications, additions and deletions are within the scope of the invention, as defined by the following claims.

What is claimed is:

1. A therapeutic mask for treatment of an eye region of a human or animal patient, the mask comprising:
at least one eye coverage portion for application to the eye region, the eye coverage portion comprising at least one receiver positioned to align with the eye; and
at least one pod for detachable placement within the at least one receiver, the at least one pod comprising configured for delivery of treatment to the eye.

2. The therapeutic mask of claim 1, comprising two independent eye coverage portions and a flexible nose bridge coupled between the two eye coverage portions.

3. The therapeutic mask of claim 1, wherein the pod contains an antimicrobial material.

4. The therapeutic mask of claim 1, wherein the receiver comprises a pocket indented from a backing of the eye coverage portion.

5. The therapeutic mask of claim 1, wherein the pod is detachably secured within the at least one receiver with hook-and-loop fastener material.

6. The therapeutic mask of claim 1, wherein the pod delivers moist heat to the eye.

7. The therapeutic mask of claim 1, wherein the pod delivers cold therapy to the eye.

8. The therapeutic mask of claim 1, further comprising a medication for delivery to the eye.

9. The therapeutic mask of claim 8, wherein the medication is impregnated into the pod.

10. The therapeutic mask of claim 8, wherein the medication is applied to a medication delivery disc for placement between the eye and the pod.

11. The therapeutic mask of claim 2, wherein the flexible nose bridge is adjustable.

12. The therapeutic mask of claim 1, wherein the mask comprises a head strap for securing to the patient.

13. The therapeutic mask of claim 12, where the head strap comprises an elastic material.

14. The therapeutic mask of claim 12, where the head strap is adjustable.

15. The therapeutic mask of claim 12, where the head strap further comprises a comfort wrap.

16. The therapeutic mask of claim 1, where the mask comprises nanobeads incorporating an antimicrobial metal.

17. The therapeutic mask of claim 1, where the pods are circular.

18. The therapeutic mask of claim 1, where the pods are different colors indicating their treatment purpose.

19. The therapeutic mask of claim 1, where the mask is constructed of a lightweight breathable foam.

20. The therapeutic mask of claim 1, where the eye coverage portion further comprises a cushioned area around the periphery of the eye coverage portion.

21. A method of treating an eye of a subject, said method comprising removably attaching a pod to a receiver portion of an eye coverage portion of the therapeutic mask, the pod comprising a moist heat delivery material, and applying the mask to an eye area of the subject with the pod held proximal to the eye by the mask to deliver moist heat therapy to the eye.

22. The method of claim 21, further comprising application of a medication material to the pod and delivery of said medication material with the moist heat therapy to the eye.

23. An ophthalmic therapy device comprising a retention strap configured for placement around a wearer's head, and first and second moist heat delivery portions attached to the retention strap, wherein positions of the first and second moist heat delivery portions relative to one another are adjustable to vary spacing between the first and second moist heat delivery portions.

24. An ophthalmic therapy device comprising a retention strap configured for placement around a wearer's head, and at least one moist heat delivery portion attached to the retention strap, wherein the at least one moist heat delivery portion comprises means for protecting a cornea of the wearer from application of excess heat.

25. The ophthalmic therapy device of claim 24, wherein the means for protecting a cornea of the wearer from application of excess heat comprises a void formed in the at least one moist heat delivery portion configured to be positioned over the cornea when the device is worn over an eye region of the wearer.

26. The ophthalmic therapy device of claim 24, wherein the means for protecting a cornea of the wearer from application of excess heat comprises a thermal shield configured to be positioned between the cornea and the at least one moist heat delivery portion when the device is worn over an eye region of the wearer.
